(19) United States
(12) Patent Application Publication
(10) Pub. No.: US 2021/0000383 A1
(43) Pub. Date: Jan. 7, 2021

(54) CAPACITIVE SENSOR

(71) Applicant: KING ABDULLAH UNIVERSITY OF SCIENCE AND TECHNOLOGY, Thuwal (SA)

(72) Inventors: Muhammad Mustafa HUSSAIN, Hercules, CA (US); Sherjeel KHAN, Thuwal (SA)

(21) Appl. No.: 16/979,936

(22) PCT Filed: Apr. 8, 2019
(86) PCT No.: PCT/IB2019/052882

§ 371 Date: Sep. 11, 2020

Related U.S. Application Data
(60) Provisional application No. 62/655,301, filed on Apr. 10, 2018.

(51) Int. Cl. A61B 5/087; H04R 1/46; H04R 19/04; B81C 1/00

(52) U.S. Cl. CPC A61B 5/087; H04R 1/46; B81B 2203/0127; B81C 1/00182; H04R 2410/03; H04R 19/04

(57) ABSTRACT

A capacitive sensor includes a sensor body having a cavity. The sensor body is non-electrically conductive. The sensor also includes a first diaphragm having a metallic conductor layer. The first diaphragm is arranged on the sensor body on a first side of the cavity. The sensor further includes a second diaphragm having a metallic conductor layer. The second diaphragm is arranged on the sensor body on a second side of the cavity. An air gap is formed in the cavity between the first and second diaphragms, the air gap having a height equal to a height of the sensor body.

### CROSS-REFERENCE TO RELATED APPLICATIONS

This application claims priority to U.S. Provisional Patent Application No. 62/655,301, filed on Apr. 10, 2018, entitled “LOW-COST WEARABLE STETHOSCOPE FOR CHRONIC MONITORING OF RESPIRATORY FUNCTION,” the disclosure of which is incorporated herein by reference in its entirety.

### BACKGROUND

### Technical Field

Embodiments of the subject matter disclosed herein generally relate to a flexible and low-cost capacitive sensor that can be used to monitor a patient's breathing.

### Discussion of the Background

The number of patients suffering from respiratory issues such as asthma is on the rise, with an estimated 334 million individuals affected as of 2014. Doctors currently rely on frequent visits from patients self-reporting symptoms of asthma and must recommend treatment without access to any real-time physiological data. Physicians and patients alike need to be equipped with a device that can continuously monitor chronic respiratory disease symptoms like asthmatic wheezing, especially since wheezing can occur any time of the day and even during sleep. Most treatments for chronic respiratory diseases rely on detecting and avoiding asthmatic triggers. However, few or no devices have been developed so far that provide long-term continuous monitoring of respiratory symptoms and are also inexpensive, easy to use, and comfortable to wear.

A patient is diagnosed with active asthma if three or more wheezing episodes occur in a year. An Asthma Detection and Monitoring study concluded that an early diagnosis of asthma is possible using noninvasive techniques by observing the airway resistance in the trachea, which produces wheezing sounds. Wheezing is characterized by musical, sinusoidal sounds superimposed on breathing at frequencies of >100 Hz and with a duration of >250 ms. Wheezing traverses through any medium by the fluctuation of pressure. Thus, wheezing can be detected by an acoustic/pressure sensor.

Several approaches have been presented for respiratory disease detection using acoustic sensors, albeit fabricated using complex and expensive technologies. These complex technologies require signal conditioning interfaces to read and interpret the data received from the sensor. Electrocardiography (ECG) is a reliable method for wearable health monitoring and wheezing detection, but the data acquisition process is complicated. The ECG sensors need complex signal conditioning circuits to convert the raw data into something meaningful, which reduces their feasibility as wearable monitors; large PCB (Printed Circuit Board) boards using several ICs (Integrated Circuits) are required to process the signal from the sensor before it can be read by a microprocessor. Even with complex electronic interfaces, the ECG signals are still prone to motion and muscle artifacts.

Soft materials have also been used to detect human vocalization using muscle movements. For example, woven graphene fabric has been used to monitor throat muscle movement in response to sounds originating in the neck. Some other flexible approaches to collecting reliable acoustic data included single-walled carbon nanotube (SWNCT) embedded in a hydrogel and nanowires grown on polytetrafluoroethylene (PTFE) films, but additional sensors were required to interpret the data. Additionally, these graphene/CNT-based sensors required complex fabrication processes that increased costs. The complexity of the data acquisition systems and high fabrication costs have hindered widespread adoption of these sensors by the healthcare industry.

Microphones have proven to be the most practical solution to acquire sounds from the neck or chest. Wheezing occurrences can be automatically detected from the sensor data using signal processing algorithms, thus increasing the likelihood of early diagnosis. An early diagnosis of asthma can help prevent the likelihood of a severe attack and patients can take medicines to prevent the oncoming attack and cease any activity that triggered the attack. High-performance MEMS (microelectromechanical) based sensors have been available for the past 15 years, but they have several failings. These sensors are rigid, making them less comfortable for wearable disease monitoring. Furthermore, to reduce the high costs of the silicon-processing equipment and the silicon itself, the sensors are small, which cause the sensors to have a very high resonance frequency (in the kHz range) and very small output signals. Therefore, they require complex signal amplification circuits, which introduce additional noise that must be reduced by signal conditioning circuits. This compromise is made because using a higher resonance frequency diaphragm presents the advantage of having higher frequency response range, which is desirable when microphones are intended for sensing human speech. However, when detecting a limited frequency range, as in case of detecting wheezing (100-1200 Hz), a larger diaphragm with lower resonance frequency is desirable in order to obtain the maximum signal-to-noise ratio.

Therefore, there is a need for inexpensive flexible acoustic sensors that can satisfactorily detect respiratory disease symptoms using sound as the input.

### SUMMARY

According to an embodiment, there is capacitive sensor, which includes a sensor body having a cavity. The sensor body is non-electrically conductive. The sensor also includes a first diaphragm having a metallic conductor layer. The first diaphragm is arranged on the sensor body on a first side of the cavity. The sensor further includes a second diaphragm having a metallic conductor layer. The second diaphragm is arranged on the sensor body on a second side of the cavity. An air gap is formed in the cavity between the first and second diaphragms, the air gap having a height equal to a height of the sensor body.

According to another embodiment, there is capacitive sensor system having a capacitive sensor, which includes a sensor body having a cavity. The sensor body is non-electrically conductive. The sensor also includes a first diaphragm having a metallic conductor layer. The first diaphragm is arranged on the sensor body on a first side of the cavity. The sensor also includes a second diaphragm having a metallic conductor layer. The second diaphragm is arranged on the sensor body on a second side of the cavity. An air gap is formed in the cavity between the first and second diaphragms, the air gap having a height equal to a height of the sensor body. The capacitive sensor system also includes a capacitance-to-digital converter configured to convert analog capacitance measurements of the capacitive sensor into digital measurements.

According to a further embodiment, there is a method for monitoring respiratory function of a patient. A capacitive sensor is attached on the patient's chest. The capacitive sensor includes a sensor body having a cavity. The sensor body is non-electrically conductive. The sensor also includes a first diaphragm having a metallic conductor layer. The first diaphragm is arranged on the sensor body on a first side of the cavity. The sensor also includes a second diaphragm having a metallic conductor layer. The second diaphragm is arranged on the sensor body on a second side of the cavity. An air gap is formed in the cavity between the first and second diaphragms, the air gap having a height equal to a height of the sensor body. The capacitive sensor outputs a signal comprising analog capacitance measurements of the capacitive sensor. A matched filter filters the signal with a predetermined signal. The matched filter outputs a signal having peaks above a noise floor responsive to the signal being sufficiently similar to the predetermined signal.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIGS. 1A-1D are schematic diagrams of capacitive sensors according to embodiments;

FIGS. 2A-2D are schematic diagrams of a method for forming a capacitive sensor according to embodiments;

FIG. 3 is a schematic diagram of a capacitive sensor system according to embodiments;

FIG. 4 is a flow diagram of a method for using a capacitive sensor according to embodiments.

### DETAILED DESCRIPTION

The following description of the exemplary embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to the terminology and structure of capacitive sensor.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

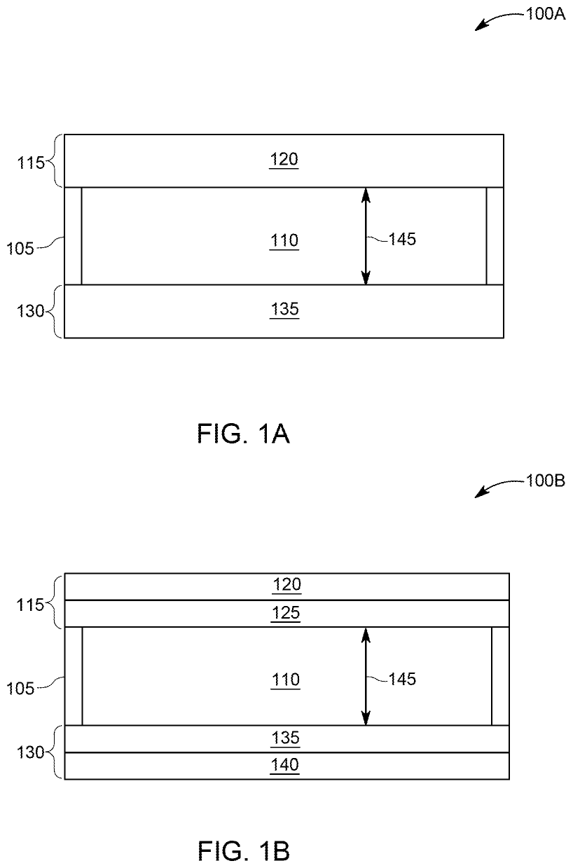

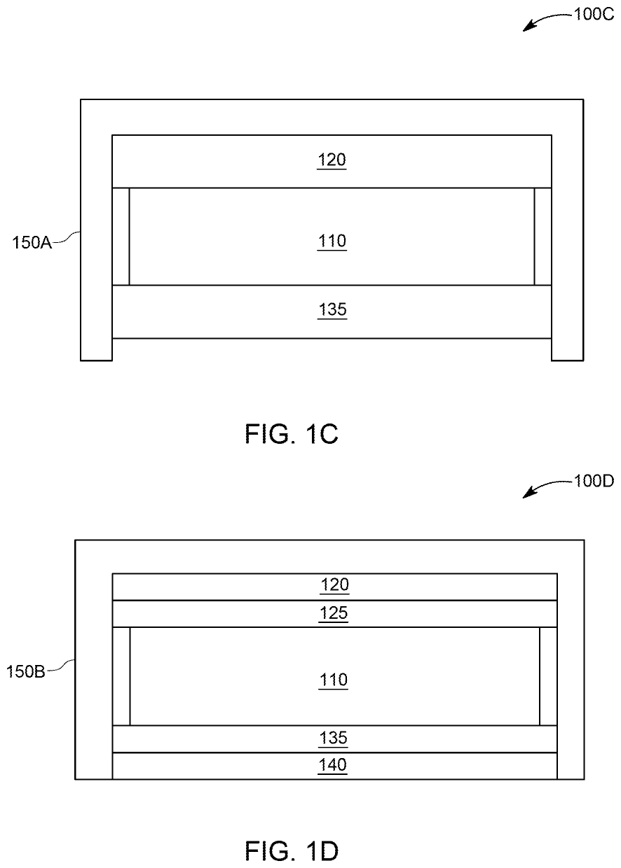

FIGS. 1A-1D are schematic diagrams of capacitive sensors according to embodiments, which can also be referred to as acoustic or pressure sensors because the capacitance of these sensors varies depending upon the pressure (either direct or via acoustic waves) impinging upon the diaphragm of the sensor.

Turning first to FIG. 1A, the capacitive sensor 100A includes a sensor body 105 having a cavity 110. The sensor body 105 is non-electrically conductive. A first diaphragm 115, comprising a metallic conductor layer 120, is arranged on the sensor body 105 on a first side of the cavity 110. A second diaphragm 130, comprising a metallic conductor layer 135, is arranged on the sensor body 105 on a second side of the cavity 110. An air gap 145 is formed in the cavity 110 between the first 115 and second 130 diaphragms. The air gap 145 has a height equal to a height of the sensor body 105. When the capacitive sensor 100A is impinged upon by sound waves, such as sounds from a patient's chest, the second 130 diaphragm will deflect into the air gap 145, which acts as a dielectric for the capacitive sensor 100A. Because the air gap 145 acts as a dielectric for the capacitive sensor 100A, the entire cavity 110 should be filled with air. Filling even a portion of the cavity 110 with something other than air, such as foam, would make the capacitive sensor 100A unsuitable for its intended purpose. In an embodiment, the metallic conductor layers 120 and 135 can be any thin metallic conductor that exhibits the desired resonance frequency such as, for example, aluminum or aluminum foil. The capacitive sensor 100A is particularly advantageous because it does not require an electrical power source. Instead, the force on the second diaphragm generates vibrations that change the output capacitance that is detected by, for example, a capacitance-to-digital converter.

As will be described in more detail below, the first 115 and second 130 diaphragms are dimensioned (i.e., the thickness and lateral dimensions of the diaphragms) so that the diaphragms have a particular resonant frequency, which depends upon the breathing conditions being monitored using the capacitive sensor 100A. With respect to detecting wheezing from the trachea, although the wheezing lies in the range of 100-2500 Hz, it is reduced to only 100-1200 Hz from the chest because lung tissue, chest wall, skin, and air absorb the higher frequencies before they reach the capacitive sensor. Therefore, when the capacitive sensor is designed to detect wheezing, the second diaphragm 130 of the sensor should resonate in the frequency range 100-1200 Hz, so that it could respond to the maximum spectrum of wheezing sounds emitted from the chest.

The capacitive sensor 100B illustrated in FIG. 1B is similar to the one in FIG. 1A and only the differences will be discussed. The capacitive sensor 100B has a first diaphragm 115 comprising a metallic conductor layer 120 arranged on a support layer 125 and a second diaphragm comprising a metallic conductor layer 135 arranged on a support layer 140. The support layers 125 and 140 increase the operational lifetime of the capacitive sensor 100B because metallic conductor is subject to plastic deformation after prolonged usage, which can affect the resonant frequency of the capacitive sensor, and thus can affect the reliability of the measurements output by the capacitive sensor.

The capacitive sensor 100C illustrated in FIG. 10 is similar to the one in FIG. 1A with the addition of an electrically insulated housing 150A. Because the capacitive sensor is intended to be attached to a patient's chest, the metallic conductor layer 135 of the second diaphragm 130 should not directly contact the patient's skin, which would affect the operation of the capacitive sensor. Thus, the second diaphragm 135 is arranged so that there is an open space between the bottom of the second diaphragm 135 and the bottom of the electrically insulated housing 150A. The electrically insulated housing 150A can also be acoustically insulating to reduce the effect of extraneous background noise from affecting the capacitive sensor. In an embodiment, the electrically insulated housing 150A comprises, for example, Styrofoam. It should be recognized, however, that other types of materials can be used for the electrically insulated housing 150A.

The capacitive sensor 100D illustrated in FIG. 1D is similar to the one in FIG. 1B with the addition of an electrically insulated housing 150B, which is similar to the electrically insulated housing 150A illustrated in FIG. 10. The difference between the electrically insulated housings 150A and 150B is that the bottom of the second diaphragm 135 can be aligned with the bottom of the electrically insulated housing 150B because the support layer 140 is arranged underneath the metallic conductor layer 135, and thus the support layer 140 electrically insulates the metallic conductor layer 135 from the patient's body.

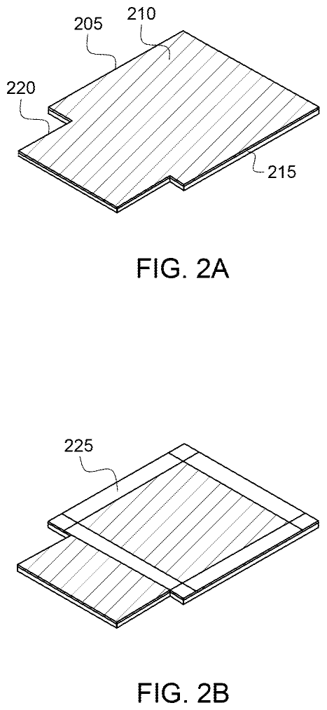

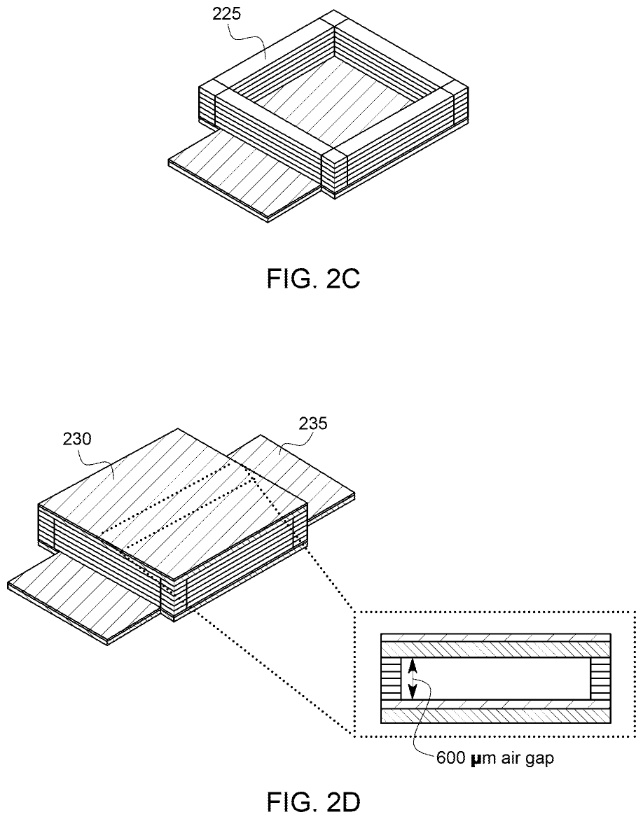

FIGS. 2A-2D are schematic diagrams of a method for forming a capacitive sensor according to embodiments. The method illustrated in FIGS. 2A-2D involve the formation of the capacitive sensor using low-cost materials. As illustrated in FIG. 2A, a diaphragm 205 comprising a support layer 210 and a metallic conductor layer 215 is formed. This diaphragm corresponds to the second diaphragm discussed above. In an embodiment, the diaphragm 205 can comprise a metallic conductor metallized film, such as the aluminum metallized polyimide film LR-PI 100AM by Liren. This aluminum metallized polyimide film is very thin, consisting of a 200 nm thick aluminum layer on top of a 25 μm thick layer of polyimide. Studies have shown that using a thin diaphragm results in increased deflection of the diaphragm, and that a decreased aluminum-on-polyimide thickness increases the overall tensile strength. Additionally, the presence of aluminum improves the linear elastic range compared to using only polyimide.

A first electrode 220 laterally extends beyond the sensor body and is electrically coupled to the diaphragm 205. The first electrode 220 can be formed separately from the diaphragm 205 and then be mechanically and electrically coupled to the diaphragm 205. Alternatively, the first electrode 220 and the diaphragm 205 can be formed from a single sheet of material (either forming them from a single sheet of aluminum and a single sheet of support material and joining the two sheets or from an integrated sheet of aluminum and support material) that is shaped (e.g., cut) to achieve the shape illustrated in FIG. 2A.

As illustrated in FIGS. 2B and 2C, the sensor body is then formed. In the illustrated embodiment this is achieved using double-sided tape 225, as illustrated in FIG. 2B. In an embodiment, each strip of the double-sided tape 225 was 1.2 mm wide and ˜90 μm thick. As illustrated in FIG. 2C, the sensor body is formed from six layers of double-sided tape 225, which forms an air gap that is greater than 550 μm. Referring now to FIG. 2D, another diaphragm 230, which corresponds to the first diaphragm discussed above, is arranged on top of the sensor body. The diaphragm 230 has the same composition as diaphragm 205. Further, a second electrode 235 laterally extends beyond the sensor body and is electrically coupled to the diaphragm 230. Specifically, a portion of the second electrode 235 runs vertically along the sensor body so that the portion of the second electrode 235 that is laterally extending beyond the sensor body is electrically coupled to the diaphragm 230. The second electrode 235 can be formed separately from the diaphragm 230 and then be mechanically and electrically coupled to the diaphragm 230. Alternatively, the second electrode 235 and the diaphragm 230 can be formed from a single sheet of material (either forming them from a single sheet of aluminum and a single sheet of support material and joining the two sheets or from an integrated sheet of aluminum and support material) that is shaped (e.g., cut) to achieve the shape illustrated in FIG. 2D.

As illustrated in the sectional view in FIG. 2D, accounting for the adhesive of the double-sided tape 225, the resulting capacitive sensor has an air gap of ˜600 μm, which occupies the entirety of the cavity formed by the sensor body and the diaphragms 205 and 230.

It should be recognized that the method illustrated in FIGS. 2A-2D is merely one way of forming the disclosed capacitive sensor and that other materials can be employed. Although the capacitive sensor illustrated in FIGS. 2A-2D employs square-shaped diaphragms, the diaphragms can have other shapes, such as rectangular or circular. In fact, testing of square-, rectangular-, and circular-shaped diaphragms demonstrated that the circular-shaped diaphragm exhibited the largest deflection, while the rectangular-shaped diaphragm exhibited the smallest deflection. A square-shaped diaphragm was employed in the capacitive sensor illustrated in FIGS. 2A-2D to demonstrate a low-cost and do-it-yourself approach that does not require the additional complexity of forming the diaphragms into a circular shape. Nonetheless, rectangular- and circular-shaped diaphragms can be employed instead of a square-shaped diaphragm, if desired.

The size of the diaphragms was also evaluated. Diaphragms with a larger surface area have been mathematically proven to result in a larger deflection. However, studies show that the resonance frequency decreases as the size of the diaphragm increases. It has also been shown that large-diaphragm condenser microphones suffer from a proximity effect as the sound intensity falls significantly with increasing distances. Because the thickness of the diaphragm also affects the deflection and resonance frequency, the reduced resonance frequency due to the increased size of the diaphragm can be at least partially accounted for by adjusting the thickness of the metallic conductor and support layers forming the diaphragms.

In order to find the optimal diaphragm size, it was investigated as to how both deflection and frequency changed as the side lengths of the square diaphragm varied from 0.5 to 3.0 cm. Consistent with similar studies, the evaluation showed that as the lateral size of the diaphragm increased, the deflection increased and the resonance frequency decreased. Experiments demonstrated that diaphragms with side lengths of 1-3 cm had a resonance frequency within the desired range for detecting a patient's wheezing. However, the largest diaphragm (3 cm sides) would interfere with the patient's everyday movements, and the smaller diaphragm (1 cm sides) had smaller deflection. Thus, in order to balance between the output signal and comfort, the diaphragm could be formed with sides having a length of 2 cm. It should be recognized that these sizes are merely exemplary and other sizes can be employed to balance the interference with the patient's everyday movements and the amount of diaphragm deflection.

As discussed above, the sounds of wheezing fall under 1000 Hz, the median frequencies lie within the range 200-400 Hz, and Computerized Respiratory Sound Analysis (CORSA) specifies dominant frequency of a wheeze to be >100 Hz and have a duration of >100 ms. Thus, when it is intended to use the capacitive sensor to detect wheezing, the diaphragm should resonate around a similar frequency. Sounds of varying frequencies (100-1000 Hz) were played at a distance of 2 mm in front of the diaphragm to determine the resonance frequency. The 2 mm distance was chosen to mimic the gap that should be maintained between human skin (a conductor) and the sensor in order to keep the capacitance of the diaphragm from changing. The frequency having the maximum amplitude was taken as the resonance frequency. A frequency sweep of a capacitive sensor with square-shaped diaphragms having a side length of 2 cm and a 600 μm air gap between the two diaphragms demonstrated that the amplitude of output escalated after 200 Hz, peaking at 250 Hz. The output remained high until 450 Hz, after it became low again. It was found that the output at 250 Hz shows the acoustic resonance pattern.

From these experiments it was concluded that the larger dimensions of the diaphragm allowed the capacitive sensor to resonate at lower frequencies than the MEMS microphones because the diaphragms of the MEMS microphones are much smaller in size and have a much higher resonance frequency, and accordingly they produce a very small deflection that consequently produces a small output signal. Accordingly, MEMS microphones require signal amplifications circuits, which introduce additional sources of noise and power consumption. In contrast, the square-shaped diaphragm having sides in the range of 1-3 cm produced sufficient displacement of the diaphragms to produce sufficiently large changes in the output signal (i.e., a large change in capacitance), and thus did not require signal conditioning or amplifications to produce a signal large enough to be easily detected by a conventional microprocessor. This allows a do-it-yourself production of a complete point-of-care device for asthma monitoring.

When the capacitive sensor is employed to detect wheezing, the sensor will be worn on the patient's chest, and thus must be able to withstand external forces other than sounds, like bending, human handling, varying temperatures, and sweat. Accordingly, the ability of the sensor to endure repeated bending and different pressure, temperature, and humidity conditions was evaluated. In order to test the performance of the capacitive sensor when bent, the capacitive sensor was subjected to 700 cycles of bending the radius of 5 mm. Bending the sensor reduced the capacitance as the air gap between the two capacitor plates decreased. However, upon releasing the structure between cycles 669 and 670, the sensor fully recovered its initial capacitance and its initial output value. This shows how the strong diaphragm materials retained their properties even when subjected to extreme bending conditions.

Furthermore, the capacitive sensor was subjected to 1,000 cycles of high pressure cyclic testing. The force applied by sound lies in the <1 Pa range, but rough human handling can reach as high as a few MPa, which means the sensor is comparatively much less likely to be affected by sound pressure than human handling. The change in capacitance for loud sounds was just a few hundred femtofarads, but that the output rose to as much as 100 pF when we subjected the sensor to a repeated force of 1 MPa. In order to test for the strength of the sensor in harsh conditions, such as pressing with a finger, the capacitive sensor was subjected to a repeated force of 1 MPa, which is equivalent to a finger poke of 60 N force on a 0.5 cm² surface area. The results of this testing confirmed that the sensor maintained its performance after hundreds of cycles. The sensor underwent a total change of 0.51 pF at the end of 1000 cycles with a standard deviation of 0.19 pF.

The temperature test involved heating the capacitive sensor from room temperature to 47° C. The capacitance of the sensor increased with temperature due to an increase in the resistance of the aluminum layer. However, the capacitance returned to its original value as the sensor cooled to room temperature.

To test resistance to sweat exposure, a sample of water with a similar salt concentration of sweat was prepared and four drops of this sample were dropped onto the capacitive sensor. The salt water drops had no effect on the output of the sensor.

After each of these tests, the sensor recovered its initial capacitance. Even when the absolute value of the capacitance changed under the various conditions, e.g., bending, high temperature, and sweat exposure, the ability to sense sounds remained unaffected. The effect of absolute change in capacitance can be accounted for by using baseline correction algorithms, such as those used with sensors that are affected by environmental conditions, to adjust the baseline value at regular time intervals.

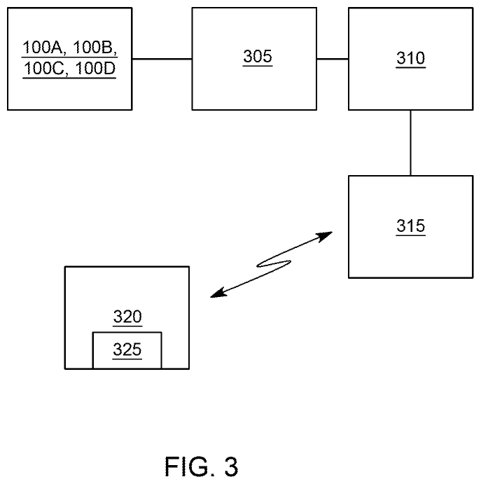

FIG. 3 is a schematic diagram of a capacitive sensor system according to embodiments. The capacitive sensor system 300 includes a capacitive sensor 100A, 1006, 100C, or 100D. The system 300 also includes a capacitance-to-digital converter 305 configured to convert analog capacitance measurements of the capacitive sensor 100A, 1006, 100C, or 100D into digital measurements. The digital measurements can be provided to a memory 310, which can be any type of memory. A wireless communication module 315 is coupled to the memory 310 and configured to periodically (and/or on demand) transmit the digital measurements to a wireless communication device 320, such as a computer, tablet, smartphone, medical diagnostic equipment, etc. The wireless communication device 320 includes a matched filter 325 to perform matched filtering on the digital measurements. Matched filters are generally used to identify a known signal or template in an unknown signal by matching the unknown signal with the known signal or template. If the matched filter output exceeds a certain threshold, the filter can determine that the signal is returned. The matched filter output produces a visible peak above the noise floor when a signal similar to the template is detected in a noisy signal. It has been found by experimentation that for a signal-to-noise ratio (SNR) of 50 or less, which is within the SNR range of signals generated by the capacitive sensor, the peaks output by the matched filter are visible enough to detect the signal among the noise using the matched filter.

The capacitive sensor 100A, 100B, 100C, or 100D, the capacitance-to-digital converter 305, memory 310, and wireless communication module 315 can all be arranged on the patient. Because the capacitive sensor 100A, 100B, 100C, or 100D does not require an electrical power source, a battery can be coupled to the capacitance-to-digital converter 305, memory 310, and wireless communication module 315 to power these devices. Although the memory 310 can be omitted, if desired, the memory 310 is particularly advantageous because it allows the powering-down of the wireless communication module 315 between periods of sending measurements instead of requiring the wireless communication module to continually send measurements.

In an embodiment, the capacitance-to-digital converter 305, memory 310, and wireless communication module 315 can be part of a single chip, such as the Bluetooth-enabled Programable-System-on-Chip (PSoC) from Cypress©. This PSoC chip is particularly advantageous because its 32-bit processor is integrated with Bluetooth-Low-Energy (BLE) 4.1 technology to achieve wireless communication with a smartphone in a total package size of 10×10×1.8 mm. BLE 4.1 has a special 1.3 μA low-power mode that consumes significantly less power than Bluetooth 2.0 and other communication protocols like Wi-Fi and ZigBee; it consumes just 10 mA instantaneous power while transmitting data at the maximum lowest connection interval of 7.5 ms. By increasing the connection interval to mere 100 ms the power consumption drops down to 0.5 mA. It operates in the 2.4 GHz ISM band with an adjustable receiver frequency of +3 to −18 dBm and a 50 meter range. Furthermore, the chip comes with 256 kB flash memory and 32 kB of RAM, so large amounts of data can be stored on-chip before sending a bulk transmission to a receiving device after every 10 seconds in order to save power. The PSoC also can be reprogrammed wirelessly by enabling the Over-the-Air (OTA) boot-loading functionality.

Researchers currently use Arduino-based modules or other primitive electronic interfaces, which are bulky, expensive, and require a wire connection for reprogramming. This makes the overall electronic interface package size too large for wearable devices. In contrast, the PSoC from Cypress allows for an extremely small, wireless solution for signal acquisition, conditioning, and transmission. It should be recognized, however, that other chips or even separate chips can be employed for the capacitance-to-digital converter 305, memory 310, and wireless communication module 315, keeping in mind that the size of the device should be small enough so as to not interfere with a patient's movements and that these components should be able to be battery-operated for a period of time over which the capacitive sensor is affixed to a patient's chest.

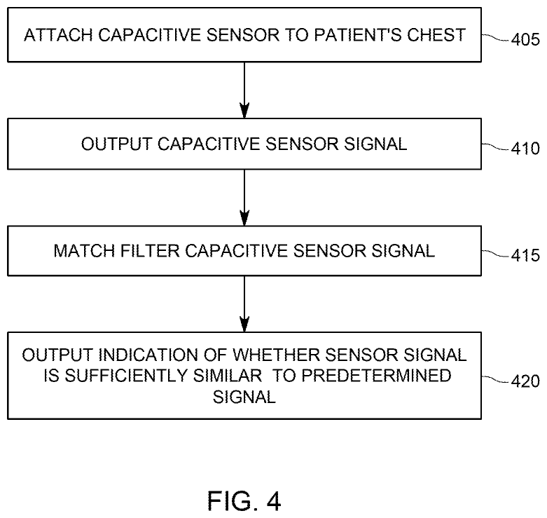

FIG. 4 is a flow diagram of a method for using a capacitive sensor according to embodiments. Initially, a capacitive sensor 100A, 100B, 100C, or 100D is attached on a patient's chest (step 405). The capacitive sensor 100A, 100B, 100C, or 100D outputs a signal comprising analog capacitance measurements of the capacitive sensor (step 410). The matched filter 325 filters the signal with a predetermined signal (step 415). The matched filter 325 outputs a signal having peaks above the noise floor responsive to the signal being sufficiently similar to the predetermined signal (step 420).

The disclosed capacitive sensor is particularly advantageous for the detection of wheezing in real time for preemptive asthma attack recognition. The sensor can be made using simple do-it-yourself methods, which are could be scaled up to large scale production. Analyses performed on the sensor confirmed that the chosen diaphragm size, material, and shape allowed it to resonate around the dominant wheezing frequency and to achieve a large deflection, thus producing a large output signal that could be directly read by a conventional microprocessor without amplification. A simple matched-filtering signal-processing technique can employed to efficiently detect wheezing, even from noisy signals. Housing the capacitive sensor in a Styrofoam box, which, together with matched filtering, significantly reduced the effect of background noise. Due to the usage of flexible materials, the sensor was non-intrusive and its placement could be customized to varying body shapes and chest sizes. Testing demonstrated that the disclosed capacitive sensor maintained its performance despite bending, repeated use, high temperatures, and sweat exposure.

Although embodiments have been described in connection with using the capacitive sensor to detect asthma-related wheezing, the capacitive sensor can be used for detecting other types of breathing conditions, and thus can be considered as a low-cost stethoscope that is wearable by the patient.

The disclosed embodiments provide a flexible and low-cost capacitive sensor. It should be understood that this description is not intended to limit the invention. On the contrary, the exemplary embodiments are intended to cover alternatives, modifications and equivalents, which are included in the spirit and scope of the invention as defined by the appended claims. Further, in the detailed description of the exemplary embodiments, numerous specific details are set forth in order to provide a comprehensive understanding of the claimed invention. However, one skilled in the art would understand that various embodiments may be practiced without such specific details.

Although the features and elements of the present exemplary embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

1. A capacitive sensor, comprising:
a sensor body having a cavity wherein the sensor body is non-electrically conductive;
a first diaphragm comprising a metallic conductor layer, wherein the first diaphragm is arranged on the sensor body on a first side of the cavity; and
a second diaphragm comprising a metallic conductor layer, wherein the second diaphragm is arranged on the sensor body on a second side of the cavity wherein an air gap is formed in the cavity between the first and second diaphragms, the air gap having a height equal to a height of the sensor body.

2. The capacitive sensor of claim 1, wherein the first and second diaphragms further comprise a support layer arranged under the metallic conductor layer.

3. The capacitive sensor of claim 2, wherein the support layer comprises polyimide.

4. The capacitive sensor of claim 1, wherein the sensor body comprises a plurality of layers of double-sided tape.

5. The capacitive sensor of claim 1, wherein a first electrode electrically coupled to the first diaphragm and a second electrode electrically coupled to the second diaphragm laterally extend beyond the sensor body.

6. The capacitive sensor of claim 1, wherein the metallic conductor comprises aluminum foil.

7. The capacitive sensor of claim 1, wherein the second diaphragm has a thickness and lateral dimensions so that the second diaphragm resonates in a frequency range of 100-1200 Hz.

8. The capacitive sensor of claim 1, wherein the capacitive sensor operates without an electrical power source.

9. The capacitive sensor of claim 1, further comprising:
an electrically insulated housing, wherein the sensor body, first diaphragm, and second diaphragm are arranged in the electrically insulated housing.

10. The capacitive sensor of claim 9, wherein the electrically insulated housing is also acoustically insulating.

11. A capacitive sensor system, comprising:
a capacitive sensor, comprising
a sensor body having a cavity, wherein the sensor body is non-electrically conductive;
a first diaphragm comprising a metallic conductor layer, wherein the first diaphragm is arranged on the sensor body on a first side of the cavity; and
a second diaphragm comprising a metallic conductor layer, wherein the second diaphragm is arranged on the sensor body on a second side of the cavity, wherein an air gap is formed in the cavity between the first and second diaphragms, the air gap having a height equal to a height of the sensor body, and
a capacitance-to-digital converter configured to convert analog capacitance measurements of the capacitive sensor into digital measurements.

12. The capacitive sensor system of claim 11, further comprising:
a wireless communication module coupled to the capacitance-to-digital converter, wherein the wireless communication module and the capacitance-to-digital converter are integrated in a common programmable-system-on-chip.

13. The capacitive sensor system of claim 12, further comprising:
a wireless communication device wirelessly coupled to the wireless communication module to receive the digital measurements, wherein the wireless communication device comprises a matched filter to filter the digital measurements.

14. The capacitive sensor system of claim 13, wherein the matched filter is configured to output amplitude peaks for the digital measurements corresponding to signals in a frequency range of 100-1200 Hz.

15. The capacitive sensor system of claim 14, wherein the second diaphragm has a thickness and lateral dimensions so that the second diaphragm resonates in a frequency range of 100-1200 Hz.

16. A method for monitoring respiratory function of a patient, the method comprising:
attaching a capacitive sensor on the patient's chest, the capacitive sensor comprising
a sensor body having a cavity, wherein the sensor body is non-electrically conductive;
a first diaphragm comprising a metallic conductor layer, wherein the first diaphragm is arranged on the sensor body on a first side of the cavity; and
a second diaphragm comprising a metallic conductor layer, wherein the second diaphragm is arranged on the sensor body on a second side of the cavity, wherein an air gap is formed in the cavity between the first and second diaphragms, the air gap having a height equal to a height of the sensor body,
outputting, by the capacitive sensor, a signal comprising analog capacitance measurements of the capacitive sensor;
filtering, by a matched filter, the signal with a predetermined signal; and
outputting, by the matched filter, a signal having peaks above a noise floor responsive to the signal being sufficiently similar to the predetermined signal.

17. The method of claim 16, further comprising:
converting the signal comprising the analog capacitance measurements into a digital signal comprising the analog capacitance measurements; and
wirelessly transmitting the digital signal to a communication device, wherein the communication device comprises the matched filter.

18. The method of claim 16, wherein the predetermined signal corresponds to wheezing in a human trachea.

19. The method of claim 18, wherein the predetermined signal is within a frequency range of 100-1200 Hz.

20. The method of claim 16, further comprising:
generating, by the capacitive sensor, the signal based on vibrations of the second diaphragm.
